(19) United States
(12) Patent Application Publication
(10) Pub. No.: US 2011/0287461 A1
(43) Pub. Date: Nov. 24, 2011

(54) ENZYMATIC ANALYTICAL MEMBRANE, TEST DEVICE AND METHOD

(75) Inventor: Qinwei Shi, Richmond Hill (CA)

(73) Assignee: ZBX CORPORATION, Toronto, ON (CA)

(21) Appl. No.: 12/994,784

(22) PCT Filed: May 26, 2009
(86) PCT No.: PCT/CA09/00684

§ 371 Date: Feb. 18, 2011

Related U.S. Application Data
(60) Provisional application No. 61/071,921, filed on May 27, 2008.

(51) Int. Cl. C12Q 1/60; C12Q 1/26; C12M 1/34; C12Q 1/28; C12Q 1/58; C12Q 1/34; C12Q 1/32

(52) U.S. Cl. 435/11; 435/18; 435/25; 435/26; 435/28; 435/12; 435/288.7

(57) ABSTRACT

The invention is directed to a novel enzymatic analytical membrane, a lateral flow enzymatic detection method, and analytical device incorporating same. The invention is useful for rapidly enzymatically determining the presence of one or more analytes in small volumes of sample. The invention provides an enzymatic analytical membrane for detecting the presence of one or more small-molecule analytes in a biological sample where the membrane comprises a receiving zone; a separation zone and a signal zone, at least one of the zones comprising one or more enzymes for converting the analytes into a form detectable by reaction with a chromogenic agent present in the signal zone and wherein the membrane horizontally receives sample at the receiving zone, and the sample continues via lateral flow through the receiving zone, separation zone and signal zone where a visible color change is formed indicating the presence of the analyte.

This application claims priority to U.S. Provisional Patent Application No. 61/071,921, filed May 27, 2008, the entirety of which is incorporated herein by reference.

### FIELD OF THE INVENTION

This invention relates to analytical membranes, methods, and devices useful for the assay of analytes in fluid samples. More specifically, the invention is directed to a novel enzymatic analytical membrane, a lateral flow enzymatic detection method, and analytical device incorporating same. The invention is useful for rapidly enzymatically determining the presence of one or more analytes in small volumes of sample.

### BACKGROUND OF THE INVENTION

Throughout this application, various references are cited in parentheses to describe more fully the state of the art to which this invention pertains. The disclosure of these references are hereby incorporated by reference into the present disclosure in their entirety.

Acetaminophen (APAP) and methanol (MeOH) overdose, if not treated immediately, can lead to serious complications and death. For these poisonings, treatment is available. Presenting symptoms may or may not be evident, making rapid identification by a laboratory method critical. Methanol measured by gas chromatography (GC) is available only in a few of the teaching hospitals. To our knowledge neither of these tests are available as a point of care test (POCT).

Immunoassay devices and procedures currently exist for detecting the presence of an analyte in a sample of biological fluid. Typically, immunochemical reactions involving antigen/antibody reactions take place on dry porous carriers such as cellular membranes through which the sample to be analyzed flows by capillary action. The presence of an analyte in the sample can be detected either visually or by using reflectance or fluorescence based detection systems and instruments. Oftentimes, the label is an enzyme label or a particulate direct label, for instance a gold sol label.

Typical immunochromatographic devices of this nature are described in the following U.S. Pat. Nos. 4,094,647; 4,235,601; 4,361,537; 4,703,017, 4,774,192; 4,839,297; 4,861,711; 4,885,240; 4,960,691; 5,075,078; 5,079,142; 5,110,724; 5,120,643; 5,135,716; 5,290,678; 5,468,648; 5,559,041; 5,591,645; 5,607,863; 5,622,871; 5,648,274; 5,656,503; 5,846,838; 5,869,345; 5,877,028; 5,998,220; 6,017,767; 6,168,956; 6,171,870; 6,187,598; 6,214,629B1; 6,228,660; 6,528,321; and 6,534,320. The Applicant's WO 2004/033101 and WO 2007/000048 also describe membrane arrays and analytical devices for the detection of analytes in samples using immunodetection.

While the aforementioned devices are generally useful for detecting certain analytes in a sample, they require the use of antibodies. Therefore, many small molecules such as methanol cannot be measured because antibodies cannot be generated against them. The detection of such small molecules can instead be carried out using an enzymatic assay format in which one or more enzymes are used to convert the molecule in question to a detectable molecule, either directly or indirectly. The quantification of the resulting molecule is a function of the concentration of the molecule being measured. The most common format for this type of assay is wet chemistry, wherein the result is analyzed with a quantification instrument, a format used mostly in central labs. Another format of the enzymatic assay is dry chemistry, wherein the enzymes and other required chemicals are dried on a solid matrix such as a dipstick. Detection of the target molecule is achieved by dipping the dipstick into the sample for a short time followed by the observation of a color change at a defined area. This type of product can only be used for urine or other non-blood sample testing, since the presence of red blood cells obscures any color change on the matrix.

U.S. Pat. No. 5,294,540 discloses a dry analytical enzymatic element for testing ethanol in blood. U.S. Pat. Nos. 6,015,683 and 6,783,731 disclose dry analytical enzymatic elements for testing acetaminophen in blood. These analytical elements detect color changes using a spectrophotometer. While effective, this is inconvenient because a sample cannot be immediately tested at a patient's bedside, instead the sample must be sent to a central lab for processing. This wastes valuable time when assessing a patient with a possible overdose. It also means that paramedics, for example, would not be able to use these devices as a POCT unless they were equipped with spectrophotometers in their ambulances.

U.S. Pat. No. 4,810,633 describes a dry analytical enzymatic test device for the determination of ethanol in an aqueous test sample such as blood or urine. In this test device, a single-layered carrier matrix is uniformly incorporated with the assay composition. The device is a matrix impregnated with a detection composition. The impregnated matrix is affixed to a support member. When a whole blood sample is tested, the impregnated carrier matrix is coated with the blood sample and excess sample must be washed or wiped off.

U.S. Pat. No. 4,900,666 describes a device similar to that of U.S. Pat. No. 4,810,633, however, it incorporates a semi-permeable membrane to filter red blood cells. After applying a blood sample to the surface of the pad it must still be washed or wiped clean to avoid confounding the color-change results. Although this method may be preferred over those listed above, it still has some inherent problems. In particular, the need to wipe or wash the excess blood from the strip increases the chance of accidental contact with blood products by medical personnel. Furthermore, washing the strip could act to effectively reduce the concentration of the sample so that the analyte would be below the level of detection. In addition, the physical process of wiping could damage the strip therefore bringing about the need for a second test and prolonging the wait for the results.

A major problem with all of the enzymatic devices listed above is that they do not automatically filter out the red blood cells from the sample. This is problematic because an accurate test result depends upon a change in color of the strip. The presence of other colored bodies, such as red blood cells, can cause confusing results.

While the aforementioned devices are generally useful for detecting an analyte in a sample, it is desirable to provide an enzymatic analytical device which is sensitive, rapid, and useful in a point of care setting. Furthermore, there is always a need for the development of a rapid blood test for such clinically relevant small molecules such as ethanol, acetylsalicylic acid, methanol, acetaminophen, homocysteine, cholesterol, urea, and combinations thereof in a one step manner that provides quick and sensitive results and only uses small sample volumes to minimize any risk of contamination to health care workers.

### SUMMARY OF THE INVENTION

The present invention is a novel lateral flow enzymatic assay device and enzymatic analytical membrane for rapidly determining the presence or absence of small molecule analytes such as ethanol, acetylsalicylic acid, methanol, acetaminophen, homocysteine, cholesterol, urea, and combinations thereof in one step with high efficiency and sensitivity in a small volume of a biological sample such as blood. The analytical membrane and device are useful as a point of care test (POCT).

According to an aspect of the present invention there is provided an enzymatic analytical membrane for detecting the presence of one or more small-molecule analytes in a biological sample, the membrane comprising in order:

a receiving zone, a separation zone and a signal zone, at least one of said zones comprising one or more enzymes for converting said analytes into a form detectable by reaction with a chromogenic agent present in said signal zone,

wherein said membrane laterally receives said sample at said receiving zone, and said sample continues via lateral flow through said receiving zone, separation zone and signal zone where a visible color change is formed indicating the presence of said analyte.

According to another aspect of the present invention there is provided a method for detecting the presence of one or more small-molecule analytes in a blood sample, comprising:

horizontally applying said blood sample to an enzymatic analytical membrane,

observing the color of said signal region,

whereby a change in color within said signal region indicates the presence of said analytes in said blood sample at or above a predetermined level.

The invention also provides enzymatic systems and kits utilizing the membranes and methods of the invention.

According to another aspect of the present invention there is provided an enzymatic analytical membrane for detecting the presence of small-molecule analytes in a biological sample, the membrane comprising:
a receiving zone having an apex at an upstream end for receiving a sample;
a signal zone downstream of the receiving zone;
an enzyme provided within at least one of said zones for converting the analyte into a detectable form; and
a chromogenic agent provided within the signal zone that produces color in the presence of the detectable form of said analyte to produce a visible color change in the membrane;
wherein the membrane is configured such that the sample flows laterally from the apex of the receiving zone to the signal zone where a visible color change is produced in the membrane in the presence of the analyte.

According to another aspect, there is provided a method for detecting the presence of small-molecule analytes in a biological sample using an enzymatic analytical membrane comprising a receiving zone having an apex at an upstream end; a signal zone downstream of the receiving zone; and enzyme provided within at least one of the zones for converting the analyte into a detectable form; and a chromogenic agent provided within the signal zone; wherein the method comprises:
applying the sample to the apex such that the sample flows laterally from the apex of the receiving zone to the signal zone; and
observing a color change in the signal zone, wherein a change in color indicates the presence of the analytes in the sample

According to another aspect, the membrane further comprises a separation zone between the receiving zone and the signal zone for filtering the sample. According to another aspect, the separation zone is glass fiber. According to another aspect, the signal zone is nitrocellulose.

According to another aspect, the sample is selected from the group consisting of whole blood, serum, plasma, urine, saliva, sweat, spinal fluid, semen, tissue lysate and combinations thereof. According to another aspect, the sample is blood.

According to another aspect, the enzyme is provided within the signal zone. In other aspects, the enzyme is provided within the separation zone.

According to another aspect, the membrane comprises a plurality of enzymes and/or a plurality of chromogenic agents, which can cooperate to detect a single analyte or can be used to detect a plurality of analytes.

According to another aspect, the detectable form of the analyte is an oxidation or reduction product of the analyte.

According to another aspect, the enzyme is selected from the group consisting of arylacylamidase, alcohol oxidase, alcohol dehydrogenase, salicylate hydroxylase, homocysteinase, cholesterol esterase, cholesterol oxidase, peroxidase, urea amidolyase, formaldehyde dehydrogenase and combinations thereof.

According to another aspect, the chromogenic agent is selected from the group consisting of o-cresol, copper sulfate, phenazine methosulfate, nitrotetrazolium blue chloride, 4-aminophenol, iodonitrotetrazolium chloride, diaphorase, NAD+, and combinations thereof.

According to another aspect, the analyte comprises acetaminophen, the enzyme comprises arylacylamidase, and the chromogenic agent comprises o-cresol. According to another aspect, the membrane further comprises copper sulfate.

According to another aspect, the analyte comprises methanol, the enzyme comprises formaldehyde dehydrogenase, and the chromogenic agent comprises nitrotetrazolium blue chloride. According to another aspect, the membrane further comprises phenazine methosulfate.

According to another aspect, the analyte comprises salicylate, the enzyme comprises salicylate hydroxylase and the chromogenic agent comprises 4-aminophenol.

According to another aspect, the analyte comprises ethanol, the enzyme comprises alcohol dehydrogenase, and the chromogenic agent comprises iodonitrotetrazolium chloride. According to another aspect, the membrane further comprising diaphorase.

According to another aspect, the membrane further comprises a control zone.

According to another aspect, there is provided an analytical device comprising the membrane described herein.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from said detailed description.

### BRIEF DESCRIPTION OF THE DRAWINGS

The present invention will become more fully understood from the detailed description given herein and from the accompanying drawings, which are given by way of illustration only and do not limit the intended scope of the invention.

FIG. 1 is a top plan view of the enzymatic analytical membrane of the invention;

FIG. 2 is a perspective view of the enzymatic analytical membrane of FIG. 1; and

FIG. 3 is an exploded view of a representative analytical device incorporating the enzymatic analytical membrane of FIG. 1.

### DETAILED DESCRIPTION OF THE PREFERRED EMBODIMENTS

The present invention is directed to a novel enzymatic analytical membrane, lateral flow enzymatic detection method and devices for enzymatically detecting one or more small-molecule analytes in a biological sample. The invention for the first time provides a rapid, sensitive, efficient and accurate enzymatic analytical membrane and method whereby a small volume of biological sample is used for application to a membrane, whereby the analyte is enzymatically converted to a detectable form.

In the invention the sample is applied to the apex of a membrane to then laterally flow through the membrane to a portion that contains enzymes therein that convert the analyte into a form detectable by selected chromogenic agents that are present in a signal zone at the other end of the membrane. The invention provides that a sample need only be applied to the apex of the membrane to continue lateral flow therethrough and provide a consistent, sensitive, and rapid detection of analyte. Because the sample is applied to one edge of the membrane (horizontally), it substantially flows in one direction, therefore minimizing wasted sample. In this manner only small sample volumes need be used. In contrast, the prior art membranes require larger sample volumes because the sample is applied to a top portion of a membrane (vertically). The sample then spreads out and down using gravity and continues laterally in any and all directions, thus wasting sample.

The present invention is also advantageous because when blood is used as the sample there is in-line red cell filtration. The present invention also provides built-in volume control; allows for capillary sampling; and is a one step, walk away, point of care test. In contrast to immunoassay membranes, the present invention involves different chemistry and reagent positioning and the membrane strip and housing design are also quite different. No prior enzymatic assay using lateral flow technology has been developed.

The enzymatic analytical membrane of the invention is constructed of a material and in a manner such that when the sample is applied to the edge of the sample receiving zone at the upstream end, the sample moves automatically, by capillary action, from the sample receiving zone downstream to the signal zone in a lateral fashion (i.e., the sample is applied horizontally to an edge of the membrane at the apex and continues flow along or through the membrane in that manner). In aspects where whole blood is used, the signal zone of the enzymatic analytical membrane has a smaller pore size than that of the remaining part of the membrane such as, for example, the separation zone so as to exclude red blood cells from entering the signal zone. In this manner, red blood cells are automatically separated from the fluid sample and never enter or overlay the signal zone. In contrast, the prior art membranes must be wiped or washed to remove the red blood cells concealing the signal zone.

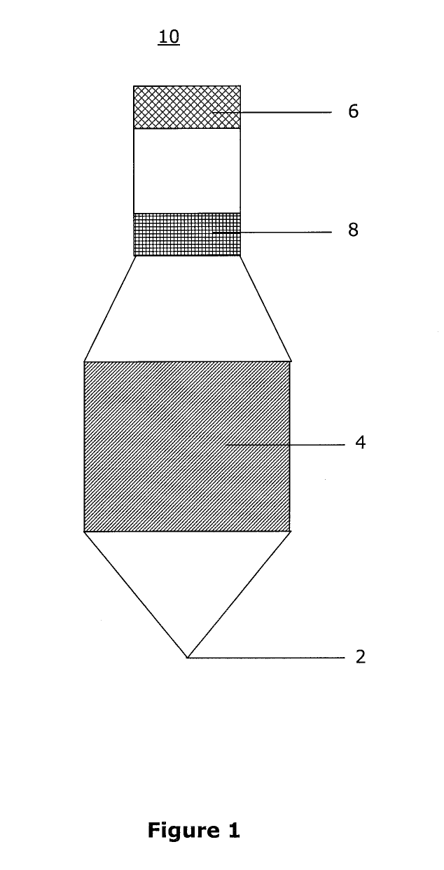

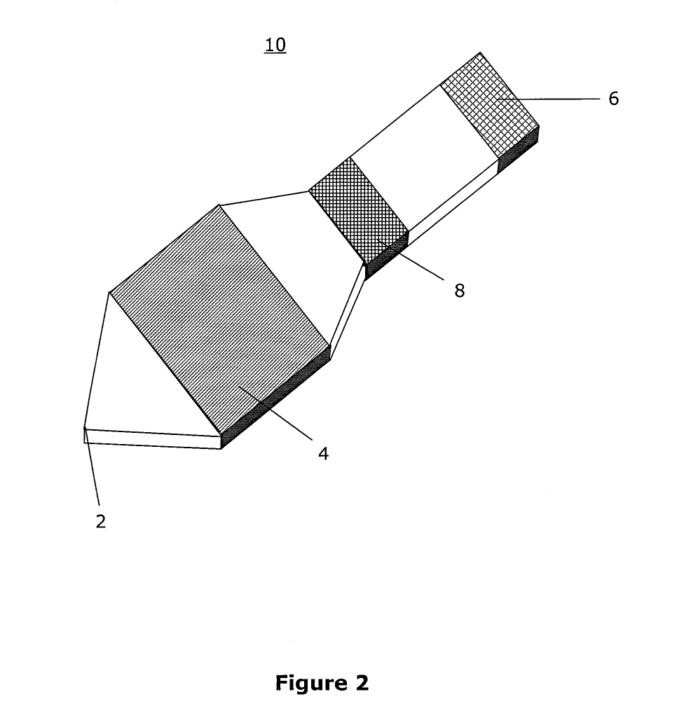

The invention is now herein described with reference to FIGS. 1 and 2 which show one embodiment of the enzymatic analytical membrane designated generally as reference numeral 10. The enzymatic analytical membrane 10 is formed from any type of porous membrane material that is body fluid compatible. The enzymatic analytical element 10 also comprises at least three zones. One of said zones is a receiving zone 2. The receiving zone 2 is located at the upstream end of the enzymatic analytical membrane. The receiving zone 2 is where sample is applied to the enzymatic analytical element 10. The sample can be applied at an edge, apex, or edge of an apex of the receiving zone 2. Downstream from the receiving zone 2 is the separation zone 4. In aspects of the invention where whole blood is used, the separation zone 4 filters the blood thus hindering the downstream movement of red blood cells. The separation zone 4 is located near the receiving zone 2 of the enzymatic analytical membrane 10 and it is generally the first zone that the fluid sample will encounter after the receiving zone 2 as it moves laterally downstream through the enzymatic analytical membrane 10. The separation zone 4 optionally contains at least one enzyme that will convert the analyte of interest into a format that is detectable in a chromogenic assay.

Downstream from the separation zone 4 is a signal zone 6. The signal zone 6 is located near the downstream end of the enzymatic analytical element 10 and is generally the last zone that the fluid sample will encounter as it moves downstream through the enzymatic analytical element 10. The signal zone 6 comprises color reagents and/or enzymes that react with the analyte(s) in the sample resulting in a visible change in color. The signal zone 6 may optionally contain a control zone 8. In aspects, the control zone 8 does not contain any specific reagents and provides a background control so that the user can compare control zone 8 with signal zone 6 to see whether there is any color development in signal zone 6 or to compare the intensity of color development within the signal zone 6 to that of the control zone 8.

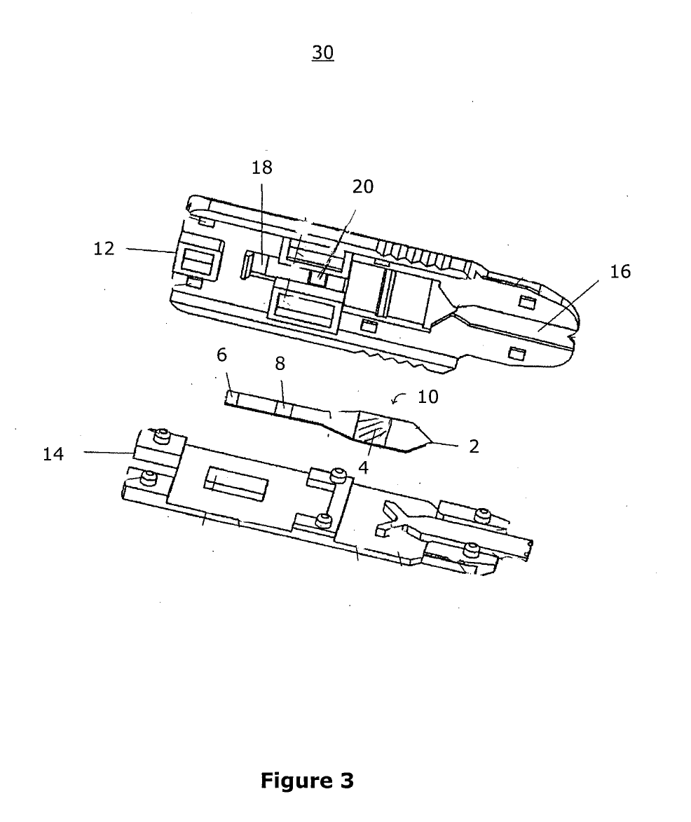

The enzymatic analytical membrane 10 of the invention can be used with any suitable device to hold the membrane and make it easier for storage, transportation and use. In one aspect, the membrane can be provided within a device, generally designated with the numeral 30 as shown in FIG. 3. Examples of such analytical devices suitable for use with the enzymatic analytical membrane 10 of the present invention are disclosed in Applicant's WO 2007/000048 patent publication (the entirety of which is incorporated herein by reference). Briefly, the analytical device 30 has an upper half 12 and a lower half 14 that cooperate to enclose the enzymatic analytical membrane 10. An indent in the bottom surface of the upper half 12 forms a sample flow channel 16. The fluid sample is applied to the sample flow channel 16. The enzymatic analytical element 10 is shaped and is placed in the analytical device 30 so that the sample enters the enzymatic analytical element 10 through the thickness of the receiving zone 2 (i.e., at an edge or apex of the receiving zone 2). The device is fabricated to have at least one viewing window 18 so as to view the signal zone 6 of the enzymatic analytical membrane 10. The device may optionally have a second viewing window 20 so as to view the control zone 8, if present. The analytical device 30 may optionally include a removable cap (not shown) that is designed to cooperate with the analytical device 30 so as to facilitate the application of a small volume of sample to the enzymatic analytical membrane 10 using a micropipette, thereby protecting the user from contamination with the fluid sample. In other embodiments the analytical device could be designed for dipping into a reservoir containing a fluid sample. Such devices could also be used to house the enzymatic analytical membrane 10 of the present invention. The enzymatic analytical membrane 10 and analytical device 30 incorporating same are easy to manufacture and do not require separate sample collection or transfer devices for capillary blood samples.

In use, in one non-limiting embodiment of the invention, a single drop of whole blood sample of sufficient quantity (up to about 50 μl, and in aspects about 10 μl to about 50 μl and any range thereinbetween) is readily obtained with a finger lancet procedure. The blood sample is brought into lateral contact with a portion of the apex of the enzymatic analytical membrane 10 at the receiving zone 2, either by direct application to the enzymatic analytical element 10 or by application to the sample flow channel 16 of the analytical device 30. If an analytical device 30 is used, it will be readily apparent to one skilled in the art that the greater capillary forces of the enzymatic analytical membrane 10 than those of the sample flow channel 16 ensure that the analytical test only begins when a sufficient volume of sample is received. The sample automatically flows laterally through the thickness of the receiving zone 2 by capillary action into and through the separation zone 4 where the red blood cells are retarded within the separation zone 4, thus separating them from the plasma. The flow of small-molecule analytes is not hindered by the separation zone 4. In the course of its flow the analytes in the sample will encounter one or more enzymes and/or reagents that will convert the analyte(s) of interest into a format that is detectable in a chromogenic assay.

It will be understood that the membrane is a three-dimensional structure having a top face, a bottom face, and side faces. Edges are found on the periphery of the membrane where the various faces meet. An edge is understood to mean the line of intersection of two surfaces. In aspects, the membrane is thin and nearly two-dimensional, having a top face and a bottom face, connected by a single peripheral edge. In other aspects, the membrane is thicker and more three-dimensional with two or more defined edges and side faces around the periphery. It is understood by one skilled in the art that the thickness of the membrane is not limiting. As is shown in the figures, in aspects the membrane is shaped such that it forms an apex at the upstream end. An apex is understood to mean an end that tapers to a point or a tip. The apex itself forms an edge, where the two tapering sides meet. The biological sample is applied at the upstream end of the membrane for lateral flow downstream through the membrane. More specifically, the biological sample is applied to the membrane at the apex or at the edge of the apex portion of the membrane.

The product continues to flow downstream by capillary action towards the signal zone 6, where it encounters a chromogenic reagent mixture. The reaction of the detectable form of the analyte(s) with the chromogenic reagent mixture results in a color change in signal zone 6, visible to the naked eye. The optional control zone 8 downstream of the signal zone 6 will remain unchanged in its color so that it can serve as a background control. In an embodiment of the invention, the signal zone 6 is shaped with a wide upstream end and a narrow downstream end so as to obtain rapid movement of the sample through the membrane segments while focusing the signal and keeping the control zone 8 distanced from the signal zone 6.

When the fluid sample has completed its capillary flow to the signal zone 6 at the downstream end of the enzymatic analytical element 10, the sample flow channel 16 is substantially empty. This arrangement serves as a control to determine and to limit the volume of the sample used in the test. In one non-limiting embodiment, the total dimensions of the enzymatic analytical element 10 are determined by the total absorption volume occupied by a single drop of blood, about 10 μl to about 50 μl.

A test cut-off may be determined clinically based on normal range and diagnostic sensitivity and specificity. The cut-off is used to optimize the product so that the color change indicates that analyte level in the sample is at or above the cut-off value. Optimization of the enzymatic assay is achieved by varying parameters such as, but not limited to, the amount of enzyme used, the buffer pH, the reagent positions on the membrane and the amount of chromogen used. For example, if the enzymatic assay is employed to merely give a yes-no indication of analyte presence, the identity and amount of chromogenic agents and enzymes can be selected to give a very large and very rapid color change with the only issue being the development of a distinctly measurable analyte-dependent color change endpoint. If the analytical membrane is employed to not only identify the presence of the analyte but also to quantify the amount of analyte present, the degree of color change should be selected based on the reagents employed and their concentrations to give a detectable variance of color change depending on the test sample analyte levels. In such a test after a suitable period for color change development the color is read and the concentration of the analyte is determined based on the level of color change.

This invention also provides a lateral flow enzymatic detection method for the rapid analysis of analytes and components of fluid samples using the enzymatic analytical membrane 10 described above. In aspects, the lateral flow enzymatic detection method is particularly suited for the rapid analysis of components of whole blood using a one step procedure with small volume fluid samples. The analysis is conducted with minimal invasiveness as only a small amount of blood, from about 10 μl to about 50 μl, is required to obtain high sensitivity detection without background interference and with minimal hemolysis. Small volumes of whole blood can readily be provided by any type of finger lancet or pin prick to the finger, for example.

In an embodiment of the invention, the enzymatic analytical membrane 10 may be optionally provided with a backing strip, otherwise known as a backing card, for support (not shown). Typically the backing card is a polystyrene tape with an appropriate adhesive that will not migrate in the enzymatic analytical membrane 10. A suitable polystyrene tape is, for example, Super White® polystyrene tape (G & L Precision Die Cutting, Inc, San Jose, Calif.) or polyester backing 3701 from Ahlstrom (PA, USA). A transparent cover tape may also be utilized over each or all of the zones 2 to 8 to inhibit evaporation of the sample. A typical transparent cover tape suitable for use with the invention is ARcare® which is a polyester film about 50 μm thick (Adhesives Research, Glenn Rock, Pa.). The enzymatic analytical membrane 10 of the present invention may be fabricated in a variety of sizes and shapes and is not limited to that specifically shown in FIGS. 1 to 3 and described above, as is understood by one of skill in the art. Furthermore, in other embodiments, in place of a control zone 8, variations in the length of the transparent cover tape over the separation zone 4 and signal zone 6 of the enzymatic analytical membrane 10 can cause the sample, when it reaches the downstream end of the signal zone 6, to evaporate in a controlled manner revealing a readily detectable signal. In aspects, the detection may be qualitative, semi-quantitative or substantially quantitative. The color intensity may be measured alternatively by a reader or spectrophotometer if desired so that a quantitative result may be obtained.

The enzymatic analytical membrane 10 is formed from any type of porous membrane material that is blood compatible and in general, body fluid compatible. Such material may be selected from, for example but not limited to, nitrocellulose, PVDF (polyvinylidene difluoride), glass fiber such as Whatman F87-14, synthetic fiber membranes such as those available from Pall Corporation (Long Island, N.Y.), polyethersulfone and pyrrolidone membranes such as those available from Spectral Diagnostics (Toronto, Canada), and polyethylene membranes such as those available from Porex Corporation (Fairburn, Ga.). One skilled in the art would understand that any similar type of such materials as disclosed herein would be suitable for use in the invention.

In other aspects of the invention, the enzymatic analytical element 10 can be made from more than one membrane. According to this embodiment, the receiving zone 2, the separation zone 4, and the signal zone 6 can be contained within different membranes. In a non-limiting embodiment, the enzymatic analytical membrane 10 as provided with more than one membrane maintains a decreasing porosity size from the first membrane at the upstream end of the enzymatic analytical membrane 10 to the last membrane at the downstream end of the enzymatic analytical membrane 10.

In aspects of the invention the pore size of the separation zone 2 may be selected from a pore size of about 8 μm to about 60 μm (and any range there-in-between). Such ranges may include but not be limited to from about 8 μm to about 10 μm, from about 8 μm to about 20 μm, from about 8 μm to about 30 μm, from about 8 μm to about 40 μm and from about 8 μm to about 50 μm. This also includes sub-ranges of these ranges. In preferred aspects of the invention, the pore size of the separation zone 4 is selected to accommodate red blood cells without substantial hemolysis. In an aspect of this invention this pore size is about greater than the size of a red blood cell up to about 8 μm or so. In aspects of the invention, the signal zone 6 is formed from any porous membrane material as is understood by one of skill in the art, such as but not limited to nitrocellulose, PVDF (polyvinylidene difluoride), Nylon and ultra-high molecular weight polyethylene. In aspects of the invention nitrocellulose is used for the signal zone 6 and is selected to have a pore size that is less than that of the separation zone 4.

Enzymes that can be used with the enzymatic analytical element 10 are selected to be compatible with the analyte that is being detected and are used to convert the analyte to a form that is detectable in a chromogenic assay. In aspects, the enzyme catalyzes a redox reaction and converts the analyte to an oxidation or reduction product. For example, arylacylamidase may be used to convert acetaminophen to p-aminophenol. Additionally, alcohol oxidase may be used to convert methanol to formaldehyde. Other examples of enzymes that could be used in certain embodiments of the present invention include alcohol dehydrogenase, salicylate hydroxylase, homocysteinase, cholesterol esterase/cholesterol oxidase, peroxidase, urea amidolyase and so on.

The following non-limiting examples of enzymes may be used to convert analytes to detectable forms:

| Analvte | Enzyme |
|---|---|
| acetaminophen | Arylacylamidase |
| acetylsalicylic acid | salicylate hydroxylase |
| ethanol | alcohol dehydrogenase; and/or |
| | alcohol oxidase |
| methanol | alcohol dehydrogenase; and/or |
| | alcohol oxidase; and/or |
| | formaldehyde dehydrogenase |
| cholesterol | cholesterol esterase; and/or |
| | cholesterol oxidase |
| homocysteine | Homocysteinase |
| urea | urea amidolyase |

In aspects, the detectable form is an oxidation or reduction product of the enzymatic reaction. Such products are readily detectable in chromogenic assays using reagents well known to a skilled person. In this way, any analyte that can be made part of an oxidation/reduction reaction can be detected on the analytical membrane described herein.

In certain embodiments, the chosen enzyme(s) are incorporated into the separation zone 4. If the separation zone 4 does not contain at least one enzyme that will convert the analyte of interest to a format that is detectable in a chromogenic assay, at least one such converting enzyme will be present within the signal zone 6. If the reaction requires more than one enzyme, all of said enzymes may be incorporated into the separation zone 4 or into the signal zone 6. In other embodiments, the enzymes may be contained separately in different regions. It is also possible that additional zones could be incorporated in the enzymatic analytical membrane 10 to contain the enzymes so that the analyte(s) in the sample encounters each enzyme in sequence, resulting in a form of the analyte that is detectable in a chromogenic assay.

If immobilization of enzymes on the membrane is desired, such as in the signal zone 6, nitrocellulose or similar substances can be used since it is known to bind protein. On the other hand if the enzyme is desired to move with the flow of the sample, such as in the separation zone 4, one can place the enzyme in glassfiber, for example.

The enzymes and other cofactors such as NAD and copper, as well as chromogenic reagents such as o-cresol in an acetaminophen enzymatic assay, for example, and nitrotetrazolium blue chloride in a methanol assay, for example, can be dispensed onto the selected zones using a precision liquid dispenser such as IsoFlow from Imagene Technology (Hanover, N.H.). The concentrations of enzymes and other required reagents are determined experimentally according to assay requirements such as sensitivity, test time and sample type. The dispensed membrane is dried at optimized conditions for the enzymes and other reagent in the membrane. The conditions for the drying process may include parameters such as temperature, humidity and time. Different drying conditions may affect test stability, sensitivity and background. Such conditions can be easily tested by a skilled person. In other embodiments of the invention, the enzyme may be provided as a solution to be added with the sample at the time of testing. In such embodiments, the analytical device, membrane, and enzyme solution can be provided separately or together in a kit.

Any of a variety of chromogenic reagent mixtures available to the skilled artisan may be utilized in the enzymatic analytical membrane 10 of the present invention. Any chromogenic agent capable of detecting an analyte of interest can be used in the membrane described herein. The signal zone 6 contains the chromogenic reagent mixtures required for the chromogenic reaction. Examples include o-cresol and copper sulfate or phenazine methosulfate and nitrotetrazolium blue chloride; 4-aminophenol for a salicylate (aspirin) assay and iodonitrotetrazolium chloride in the presence of diaphorase and NAD+ for an ethanol assay. The enzymatic analytical membrane of the invention is suitable for use for the detection of a wide variety of analytes such as but not limited to ethanol, acetylsalicylic acid, methanol, acetaminophen, homocysteine, cholesterol, urea, and combinations thereof.

It is within the scope of the present invention to detect an analyte or even multiple analytes in the fluid sample at one time. Accordingly, it will be appreciated by one skilled in the art that one or more enzymes and/or one or more chromogenic reagents can be deposited on the enzymatic analytical membrane 10 of the present invention. The enzymes and chromogenic reagents can be selected to produce a distinct color change in the presence of each of the chosen analytes. Alternatively, the enzymes and chromogenic reagents can be selected to produce a single color change in the presence of any one of the chosen analytes. Where more than one analyte is being detected, it is possible that some color product may be insoluble. In embodiments where more than one analyte will be tested, 1) the reagents for one analyte may be placed upstream of the reagents for the other analyte; 2) the membrane may be designed so that it allows sample to be applied to one separation zone at the center of the strip and sample will flow laterally in both directions, each with a signal zone and a set of different reagents; or 3) two different tests could be placed back to back in one housing and connected with one sample channel. Other similar means for detecting more than one analyte simultaneously are contemplated and would be understood by a skilled person.

Biological samples suitable for application to the analytical membrane of the invention may include but not be limited to whole blood, serum, plasma, urine, saliva, sweat, spinal fluid, semen, tissue lysate and combinations thereof.

Rapid and accurate diagnoses based on the presence of one or more small-molecule analytes in a fluid sample, using small volumes of the fluid sample are provided by the present invention. Sensitivity requirements for a qualitative test are determined clinically. Some tests require high cut-off others require lower cut-off depending on normal range, test specificity and clinical utility. The method allows one skilled in the art to manipulate parameters such as reagent concentration, dispensing rate, location, flow rate and alternative enzyme or reagent to achieve the sensitivity/cut-off required. Test time is generally between 5-15 minutes and in aspects is less than 5 minutes, 4 minutes, 3 minutes, 2 minutes, or 1 minute. Once developed the test result is relatively stable for a long time period.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Evaluation of the Enzymatic Analytical Membrane for the Detection of Acetaminophen (APAP) and Methanol (MeOH)

Twelve sera from two different MeOH overdoses and 17 sera that were positive for APAP by immunoassay were analyzed by the enzymatic analytical membrane described herein. In addition to the commercially available quality control samples, six and ten negative sera for MeOH and APAP respectively, were also analyzed. Cross reactivity to ethanol was checked with 40, 80 and 120 mmol of ethanol. The testing technician was blind to the original results.

MeOH: Nine of the eleven POCT positive samples were positive with the gas chromatography (GC) results. Reading taken at two minutes. Two that were positive by the enzymatic analytical membrane but negative by GC were just below the detection limit of the GC suggesting that the enzymatic analytical membrane may be more sensitive than GC. The detection limit of the GC is approximately 2 mM. There was concordance with the remaining samples. Methanol did not give false positive results for the enzymatic analytical membrane.

APAP: All patient and quality control samples were in concordance with the original test results. Readings were taken at 7 minutes with a detection limit at 200 μmol.

This example demonstrates that methanol and acetaminophen enzymatic analytical membranes can be successfully used in the emergency department to detect methanol and acetaminophen in blood and therefore to triage the poisoned patient.

### Example 2

### An Acetaminophen Enzymatic Analytical Membrane Device

An acetaminophen test device using one drop of whole blood sample was prepared according to the present invention. For the signal zone, nitrocellulose (Millipore) with stated flow rate of 180 sec/4 cm was impregnated with color reagent using a conventional liquid dispenser. Color reagent contains 3% o-cresol, 6.4 mM copper sulfate and 1 M sodium hydroxide. Impregnated nitrocellulose was placed at room temperature at less than 20% relative humidity for 30 minutes. The separation zone (Whatman) was sprayed with enzyme solution and then freeze dried to remove the water. The enzyme solution contains 250 U/mL arylacylamidase (GDS Technology). The enzymatic analytical membrane was supported by polystyrene backing tape (G & L Precision Die Cutting, Inc.). The shape of the enzymatic analytical membrane was obtained using a die-cutting tool. The enzymatic analytical membrane was housed in an analytical device as shown in FIG. 3. Testing of this analytical device using 40 μL of blood or serum demonstrated excellent plasma separation and sample flow in a testing procedure requiring approximately 10 minutes. The test achieved a sensitivity of 200 μM of acetaminophen.

### Example 3

### A Methanol Enzymatic Analytical Membrane Device

A methanol test device using one drop of whole blood sample was prepared according to the present invention. For the signal zone, nitrocellulose (Millipore) with a stated flow rate of 180 sec/4 cm was impregnated with color reagent, 18 U/mL formaldehyde dehydrogenase (Sigma-Aldrich), and 6 mM β-nicotinamide adenine dinucleotide hydrate (β-NAD). Color reagent contains 0.3 mM phenazine methosulfate, 1.2 mM nitrotetrazolium blue chloride, and 0.5% Triton X-100. The separation zone (Whatman) was sprayed with 315 U/mL alcohol oxidase solution (Sigma-Aldrich). Impregnated membranes were placed at room temperature at less than 20% relative humidity for 30 minutes. The enzymatic analytical membrane was supported by polystyrene backing tape (G & L Precision Die Cutting, Inc.). The shape of the enzymatic analytical membrane was obtained using a die-cutting tool. The enzymatic analytical membrane was housed in an analytical device as shown in FIG. 3. Testing of this analytical device using 40 μL serum indicated a sensitivity of 3 mM of methanol at a testing time of approximately 3 minutes.

1. An enzymatic analytical membrane for detecting the presence of small-molecule analytes in a biological sample, the membrane comprising:
a receiving zone having an apex at an upstream end for receiving a sample;
a signal zone downstream of the receiving zone;
an enzyme provided within at least one of said zones for converting the analyte into a detectable form; and
a chromogenic agent provided within the signal zone that produces color in the presence of the detectable form of said analyte to produce a visible color change in the membrane;
wherein the membrane is configured such that the sample flows laterally from the apex of the receiving zone to the signal zone where a visible color change is produced in the membrane in the presence of the analyte.

2. The membrane of claim 1, further comprising a separation zone between the receiving zone and the signal zone for filtering the sample.

3. The membrane of claim 1 or 2, wherein the sample is selected from the group consisting of whole blood, serum, plasma, urine, saliva, sweat, spinal fluid, semen, tissue lysate and combinations thereof.

4. The membrane of claim 3, wherein the sample is whole blood.

5. The membrane of any one of claims claim 1 to 4, wherein the enzyme is provided within the signal zone or within the separation zone.

6. (canceled)

7. The membrane of any one of claims claim 1 to 6, comprising a plurality of enzymes.

8. The membrane of any one of claims claim 1 to 7, comprising a plurality of chromogenic agents.

9. The membrane of any one of claims claim 2 to 8, wherein the separation zone is glass fiber.

10. The membrane of any one of claims claim 1 to 9, wherein the signal zone is nitrocellulose.

11. The membrane of any one of claims claim 1 to 10, wherein the detectable form is an oxidation or reduction product of the analyte.

12. The membrane of any one of claims claim 1 to 11, wherein the enzyme is selected from the group consisting of arylacylamidase, alcohol oxidase, alcohol dehydrogenase, salicylate hydroxylase, homocysteinase, cholesterol esterase, cholesterol oxidase, peroxidase, urea amidolyase, formaldehyde dehydrogenase and combinations thereof.

13. The membrane of any one of claims claim 1 to 12, wherein the chromogenic agent is selected from the group consisting of o-cresol, copper sulfate, phenazine methosulfate, nitrotetrazolium blue chloride, 4-aminophenol, iodonitrotetrazolium chloride, diaphorase, NAD+, and combinations thereof.

14. The membrane of any one of claims claim 1 to 13, wherein the analyte comprises acetaminophen, the enzyme comprises arylacylamidase, and the chromogenic agent comprises o-cresol.

15. The membrane of claim 14, further comprising copper sulfate.

16. The membrane of any one of claims claim 1 to 15, wherein the analyte comprises methanol, the enzyme comprises formaldehyde dehydrogenase, and the chromogenic agent comprises nitrotetrazolium blue chloride.

17. The membrane of claim 16, further comprising phenazine methosulfate.

18. The membrane of any one of claims claim 1 to 17, wherein the analyte comprises salicylate, the enzyme comprises salicylate hydroxylase and the chromogenic agent comprises 4-aminophenol.

19. The membrane of any one of claims claim 1 to 18, wherein the analyte comprises ethanol, the enzyme comprises alcohol dehydrogenase, and the chromogenic agent comprises iodonitrotetrazolium chloride.

20. The membrane of claim 19, further comprising diaphorase.

21. The membrane of any one of claims claim 1 to 20, further comprising a control zone.

22. An analytical device comprising the membrane of any one of claims claim 1 to 21.

23. A method for detecting the presence of small-molecule analytes in a biological sample using an enzymatic analytical membrane comprising a receiving zone having an apex at an upstream end; a signal zone downstream of the receiving zone; an enzyme provided within at least one of the zones for converting the analyte into a detectable form; and a chromogenic agent provided within the signal zone of claim 1; wherein the method comprises:
applying the sample to the apex such that the sample flows laterally from the apex of the receiving zone to the signal zone; and
observing a color change in the signal zone, wherein a change in color indicates the presence of the analytes in the sample.

24. The method of claim 23, wherein the membrane further comprises a separation zone between the receiving zone and the signal zone and the method further comprises filtering the sample.

25. The method of claim 23 or 24, wherein the sample is selected from the group consisting of whole blood, serum, plasma, urine, saliva, sweat, spinal fluid, semen, tissue lysate and combinations thereof.

26. The method of claim 25, wherein the sample is blood.

27. The method of any one of claims claim 23 to 26, wherein the sample is applied in a volume of up to about 50 μl selected from up to about 10 μl; about 10 μl and from about 10 μl to 50 μl.

28. (canceled)

29. (canceled)

30. The method of any one of claims claim 23 to 29, wherein the color change occurs in a time selected from about 1 to 10 minutes after the sample is applied or; in about 5 to 10 minutes after the sample is applied or; in less than about 1 minute after the sample is applied.

31. (canceled)

32. (canceled)

33. The method of any one of claims claim 23 to 32, wherein the enzyme is provided within the signal zone, or within the separation zone.

34. (canceled)

35. The method of any one of claims claim 23 to 34, comprising a plurality of enzymes and/or a plurality of chromogenic agents.

36. (canceled)

37. (canceled)

38. (canceled)

39. The method of any one of claims claim 23 to 38, wherein the detectable form is an oxidation or reduction product of the analyte.

40. The method of any one of claims claim 23 to 39, wherein the enzyme is selected from the group consisting of arylacylamidase, alcohol oxidase, alcohol dehydrogenase, salicylate hydroxylase, homocysteinase, cholesterol esterase, cholesterol oxidase, peroxidase, urea amidolyase, formaldehyde dehydrogenase and combinations thereof.

41. The method of any one of claims claim 23 to 40, wherein the chromogenic agent is selected from the group consisting of o-cresol, copper sulfate, phenazine methosulfate, nitrotetrazolium blue chloride, 4-aminophenol, iodonitrotetrazolium chloride, diaphorase, NAD+, and combinations thereof.

42. The method of any one of claims claim 23 to 41, wherein the analyte comprises acetaminophen, the enzyme comprises arylacylamidase, and the chromogenic agent comprises o-cresol.

43. The method of claim 42, the membrane further comprising copper sulfate.

44. The method of any one of claims claim 23 to 43, wherein the analyte comprises methanol, the enzyme comprises formaldehyde dehydrogenase, and the chromogenic agent comprises nitrotetrazolium blue chloride.

45. The method of claim 44, the membrane further comprising phenazine methosulfate.

46. The method of any one of claims claim 23 to 45, wherein the analyte comprises salicylate, the enzyme comprises salicylate hydroxylase and the chromogenic agent comprises 4-aminophenol.

47. The method of any one of claims claim 23 to 46, wherein the analyte comprises ethanol, the enzyme comprises alcohol dehydrogenase, and the chromogenic agent comprises iodonitrotetrazolium chloride.

48. The method of claim 47, the membrane further comprising diaphorase.

49. The method of any one of claims claim 23 to 48, further comprising comparing the color of the membrane to a control.

50. The method of claim 49, wherein the control is a zone within said membrane.

51. (canceled)

52. (canceled)

53. An enzymatic analytical membrane for detecting the presence of one or more small-molecule analytes in a biological sample, the membrane comprising:
a receiving zone, a separation zone and a signal zone, at least one of said zones comprising one or more enzymes for converting said analytes into a form detectable by reaction with a chromogenic agent present in said signal zone,
wherein said membrane horizontally receives said sample at said receiving zone, and said sample continues via lateral flow through said receiving zone, separation zone and signal zone where a visible color change is formed indicating the presence of said analyte.

54. (canceled)
